(19) United States
(12) Patent Application Publication
(10) Pub. No.: US 2005/0232953 A1
(43) Pub. Date: Oct. 20, 2005

(54) Microemulsions having a binary phase differentiability and active substance differentiability, the production thereof and their use, particularly for the topical supply of oxygen

(76) Inventors: Wolfgang Barnikol, Mainz (DE); Alexander Teslenko, Hagen (DE)

Correspondence Address: WILLIAM COLLARD;COLLARD & ROE, P.C., 1077 NORTHERN BOULEVARD, ROSLYN, NY, 11576, US

(21) Appl. No.: 10/518,667

(22) PCT Filed: Jun. 6, 2003
(86) PCT No.: PCT/EP03/05948

§ 371 Date: Dec. 17, 2004

(30) Foreign Application Priority Data
Jun. 18, 2002 (DE) 102 26 990.4

(51) Int. Cl. A61K 38/05; A61K 31/56; A61K 9/00; A61K 35/78

(52) U.S. Cl. 424/400; 424/401; 424/725.1; 514/18; 514/171; 514/454

(57) ABSTRACT

Micro-emulsions having a binary phase differentiability and active substance differentiability, the production thereof, and their use, particularly for the topical supply of oxygen The present invention relates to skin-compatible micro-emulsions that are suitable for the treatment of hair and skin, based on a primary W/O micro-emulsions that are converted into both a secondary W/O and into a secondary O/W micro-emulsion, and can contain, in particular, both water-soluble and fat-soluble active substances in stable form. Preferably, the emulsion contains an oxygen binder such as hemoglobin, with which bound bioavailable oxygen, preferably together with other active substances, can be introduced into the skin by means of topical application, in order to support the cell growth of the stratum germinativum. These emulsions can be produced easily, without great technical effort, and can be used both in cosmetics and in medicine (dermatology).

### OBJECT OF THE INVENTION

The present invention relates to skin-compatible micro-emulsions that are suitable for the treatment of hair and skin, based on a primary W/O micro-emulsions that are converted into both a secondary W/O and into a secondary O/W micro-emulsion, and can contain, in particular, both water-soluble and fat-soluble active substances in stable form. Preferably, the emulsion contains an oxygen binder such as hemoglobin, with which bound bioavailable oxygen, preferably together with other active substances, can be introduced into the skin by means of topical application, in order to support the cell growth of the stratum germinativum. These emulsions can be produced easily, without great technical effort, and can be used both in cosmetics and in medicine (dermatology).

### STATE OF THE ART

Micro-emulsions are macroscopically homogeneous, optically isotropic, and thermodynamically stable two-phase systems that consist of two non-miscible liquids (as a rule, water and one or more non-polar organic liquids not miscible with water, generally referred to as “oil”). Furthermore, they contain surfactants. In the simplest case, micro-emulsions already form from these three components, but frequently, a co-surfactant (e.g. a short-chain aliphatic alcohol) is required. They can have water as the continuous phase (O/W micro-emulsion) or oil as the continuous phase (W/O micro-emulsion).

Micro-emulsions possess the unique property that the surface tension between the phases is very low. As a result, the phases are very stable against phase separation, thermodynamically; therefore the micelles are able to exist in the form of very small particles having a size of 20 to 200 nm (in contrast to usual emulsions having a particle size of 1 to 20μ) /P. Kumar, K. L. Mittal (Eds.), Handbook of Microemulsions. Marcel Dekker Inc., NY, 1999/.

The known micro-emulsions are formulated as a function of the active substance property. They have a broad area of application, such as petroleum extraction, the sector of technical and household cleaners, as well as the micro-dispersion of chemical substances, for example pesticides and fungicides, and more. Another potential area of use is their use as a reaction medium for chemical or enzymatic reactions between water-soluble reactants and non-polar organic compounds. Micro-emulsions are furthermore an ideal transport vehicle for peroral, intracutaneous, or transcutaneous administration of medications for humans and animals.

For example, O/W micro-emulsions that contain 0.5 to 30 wt.-% of an oil phase, together with 0.05 to 5% of a non-ionic surfactant, 0.1 to 10% of an emulsifier, as well as phospholipids and active substances not soluble in water, such as diclofenac, are described in U.S. Pat. No. B 6,113,921. By means of using the high-pressure homogenizer APV-Gaulin at a pressure of 800 bar and at elevated temperature, the particles have a size of 10 to 500 nm. Furthermore, a method for the production of compositions is known, whereby a mixture of glyceride surfactants having an HLB value of ≦16 and propylene glycol esters or polyglycerol esters are mixed with an oil phase and an aqueous phase and an active substance. Upon contact with a biological liquid, e.g. gastric juice, a micro-emulsion is formed in situ, see U.S. Pat. No. 6,309,665. U.S. Pat. No. B 5,646,109 (EP B 0746 331) relates to convertible W/O micro-emulsions to be used orally or intravenously, having an active substance soluble in water and up to 70% of a surfactant mixture of at least one C₉₋₁₃ monoglyceride having an HLB value of ≦8 and a surfactant having an HLB value of ≧8.

EP B 0580 778 describes a W/O emulsion having an active substance soluble in water and surfactants having HLB values from 7 to 14, whereby a C₇₋₅₅ propylene glycol diester is necessarily contained in the oil phase.

WO 02/09671 discloses a mixture of poloxamer block copolymer with C₈₋₁₂ fatty acids such as sodium laurate, for the emulsification of pharmacological active substances not soluble in water (analgesics, anti-depressives, immunosuppressives, anti-neoplastics, etc.). Micro-emulsions on the basis of cationic surfactants for use in the sector of hair are described according to WO 00 06 690.

Chang Il Hong et al., U.S. Pat. No. B 6,063,762 and U.S. Pat. No. B 6,306,434, have described a pharmaceutical composition containing cyclosporine, for oral application. Here, cyclosporine is mixed with an oil and a surfactant, and then reacted with a polycarbonate or polyol (Eudragit®), in dry manner.

U.S. Pat. No. B 6,191,105 describes a composition for oral administration of an insulin/hexanic acid conjugate for the treatment of diabetes. Here, an O/W surfactant is present in the aqueous phase and 30 to 90% of an oil phase.

Furthermore, micro-emulsions and micelles were used intravenously, among other things for the treatment of brain tumors, see WO 02/09671.

According to U.S. Pat. No. A 6,191,105, compositions that can be administered intramuscularly are described for the gene-therapy treatment of various illnesses, whereby polynucleotides such as RNA and DNA are present in the form of an aqueous dispersion of less than 0.1% poloxamer/polyacrylate copolymers.

In comparison with these pharmaceutical micro-emulsions, the micro-emulsions developed for cosmetic purposes are not supposed to achieve any systemic effect, i.e. they are merely supposed to penetrate to the basal membrane of the skin epithelium or to the upper layer of the dermis. This skin layer, the stratum corneum, is formed by dead keratinocytes that scale off over the course of time. The entire process from mitosis to the death of the cells and their scaling off normally takes about four weeks. The stratum corneum makes a decisive contribution to the intactness of the skin, because of its barrier functions.

With increasing age, the mitotic process slows down; according to current knowledge, this happens as early as the age of thirty. This results in an ever thinner skin, having a reduced ability to survive. Skin aging is genetically caused, for one thing. Furthermore, an insufficient supply of nutrients and building blocks, as well as numerous environmental influences, particularly sunlight, have a negative effect on the skin. Oxygen plays a decisive role in this connection.

In order to support the supply to the skin, the substances mentioned above should be delivered all the way to the cells of the skin epithelium. A prerequisite for this is the topical accessibility to the vital cells of the skin epithelium. While the mucous membranes can be reached directly, by means of local therapy, the vital cells of the skin epithelium are protected from the outside by the stratum corneum, having a thickness of 10-20 μm, which is difficult to penetrate. If an active substance is to reach the vital cells of the skin epithelium, this can only be done using a suitable vehicle that makes it possible to overcome the stratum corneum barrier. In this regard, at least two steps are differentiated, namely:
(1) penetration: entry of the substance into the stratum corneum,
(2) permeation: the diffusion of a substance from the stratum corneum into the living epidermis.

These processes, which are also significant for the medical use of topical agents as described above, must take place both in cosmetic skin care, namely the strengthening of the natural function of the skin as a barrier against environmental influences and against the loss of substances inherent to the body (e.g. water, natural fats), and in cosmetic treatment, namely the support of the vital cells with essential nutrients and therefore the restoration of integral parts of the skin that have become weak in their function, e.g. by means of promoting the biosynthesis of collagen and hyaluronic acid.

For this purpose, traditional cosmetic preparations such as creams, lotions, gels, and liposomes are used, along with physical methods such as ultrasound, electrophoresis, or iontophoresis, which are often technically complex and vary in their effectiveness. As compared with these, micro-emulsions have the advantage of the smaller particle size, in other words better permeation and a lower required amount of active substance, and they are more stable, particularly in comparison with liposomal products.

A number of micro-emulsions are known in cosmetics:

According to WO 98/15254 (EP 0930 866), W/O micro-emulsions are described for various cosmetic preparations such as lotions, shower lotions, aftershave lotions, deodorant spray, anti-acne gels, sunscreens (UV filters), deodorants, water-resistant eye make-up, and other cosmetic preparations based on W/O emulsifiers, which necessarily contain cross-linking agents such as dimethicon copolyols.

U.S. Pat. No. B 6,315,989 relates to a composition containing hydrogen peroxide as a W/O micro-emulsion for dyeing hair, which contains not only the oil phase and the aqueous phase but also 1-65% of an organic surfactant having an HLB value of 12-16.

Micro-emulsions containing UV filters, for protecting the skin against the rays of the sun, are described in the patents U.S. Pat. No. B 6,207,140, U.S. Pat. No. B 5,876,702, and EP A 1 092 414, which particularly contain lipophilically modifiable emulsifiers that are dependent on pH or temperature. In this connection, sulfonated UV filters are used, in particular, which act as electrolytes and thereby also influence the lipophilia of the emulsifiers.

In the patent U.S. Pat. No. B 5,389,607, alcohol-free, perfume-containing micro-emulsions having a surfactant based on polyethylene glycol, together with a polyglycerol and a phosphate ether, are described.

From this state of the art, it is evident that the topical use of micro-emulsions is primarily aimed at an effect on the skin surface. The micro-emulsions described above do not demonstrate any deep penetration into the stratum corneum or the dermis. Furthermore, they are either complicated in production (e.g. only at high pressure or in situ), or very high proportions of surfactant are required. Finally, emulsions already described only act on mucous membranes.

DE A 1 44 11 557 relates to a method for the production of W/O emulsions, in that at least 30% of a C₁₂₋₂₄ dialkyl ether, 10 to 35% of a lipophilic emulsifier having an HLB value of 6 to 10 (W/O emulsifier), and 1 to 10% of a hydrophilic (O/W) emulsifier having an HLB value >11 are used as the oil component. Thus, there is always an excess of W/O to O/W emulsifier in connection with dialkyl ethers as the oil component.

In WO 94/26234, W/O emulsions are described that necessarily contain 25 to 85% petrolatum and 15 to 40% solid waxes, as well as maximally 5% water, in order to be able to be used as lip balm formulations. Accordingly, the formulations are produced in heat, so that the wax can be sufficiently melted.

U.S. Pat. No. A 4,797,273 relates to a W/O micro-emulsion that necessarily has a polysiloxane compound. This is supposed to be particularly effective for dry skin.

In U.S. Pat. No. A 4,797,272, usual W/O micro-emulsions for keeping the skin moist are described; they contain usual surfactants and various moisturizing components.

In WO A 00/61083, transparent micro-emulsions are disclosed, whereby the ratio of surfactant(s) to oil component(s) is 2:1 to 1:1. The composition is produced with the application of heat (75° C. to 80° C.).

### TASK OF THE INVENTION

The task of the present invention is therefore to make available an agent with which the skin or the hair can be effectively supplied, from the outside, with the necessary cellular nutrients, in a sufficient amount, whereby in particular, a supply of oxygen is to be provided and the stratum corneum as a barrier is supposed to be overcome. In this connection, there is to be no restriction to active substances that are only soluble in water or only soluble in oil (fat).

Furthermore, the agent is supposed to be easy to produce and the amount of surfactant is supposed to be as low as possible. Another purpose of the invention is to effectively treat skin, in particular degenerative skin that has been damaged or modified by means of external or immunologically related influences, both cosmetically and dermatologically/pharmaceutically.

In particular, a supply of oxygen is to be provided in such a manner that no excessive or harmful supply, such as in the case of oxygen administered in gaseous form, will occur, as this can have a toxic effect. This also includes effective penetration, which is not possible with normal emulsions, as explained, see the composition of vitamin, glucose, and hydrogen peroxide in the form of an O/W emulsion as a cream known from U.S. Pat. No. B 5,380,764, whereby here, there is not only the problem of poor penetration, but also the problem of the physically active oxygen, which can damage the tissue, see above.

The agent is furthermore supposed to be easy to use as such, and therefore particularly be liquid or in gel form, to be rubbed in, particularly also micro-sprayable, but without requiring special substances such as gel forming agents.

### SOLUTION FOR THE TASK

These tasks are accomplished, according to the invention, in that a micro-emulsion is made available, which contains not only an oil phase and an aqueous phase but also a system of W/O and O/W surfactants in a ratio of 1:4 to 1:1.2, together with small amounts of emulsifiers and, if necessary, low alcohols. Surprisingly, such micro-emulsions have nano-micelles, without specific cross-linking agents such as dimethicon being required. Furthermore, at the same time, both water-soluble and fat-soluble active substances can be incorporated, without instability occurring. Finally, the micro-emulsion prepared in this manner can be converted to a secondary W/O micro-emulsion by reaction with an aqueous phase, or, at an elevated water content, into a secondary O/W micro-emulsion.

The emulsion according to the invention is therefore differentiable both with regard to the active substances and with regard to the desired phase, in binary manner.

With such preparations, the skin/hair of mammals, particularly of humans, can be treated cosmetically, dermatologically, or also pharmaceutically/medically, in simple manner, whereby the emulsion can also be produced in simple manner.

The micro-emulsion according to the invention is primarily a water-in-oil emulsion, with binary phase differentiability and active substance differentiability, and particularly comprises
a) 45 to 90 wt.-% of a liquid oil phase;
b) 0.1 to 45, particularly 5 to 40 wt.-% of a mixture of one or more W/O and one or more O/W surfactants, in a ratio of 1:4 to 1:1.2;
c) 0.01 to 20 wt.-% of one or more emulsifiers;
d) 0.00 to 15 wt.-% of one or more monovalent or bivalent C₁₋₈ alcohols; and
e) 1 to 10 wt.-% water or aqueous solutions,
   whereby the micelles of the primary micro-emulsion have a particle size of 20 to 400 nm, particularly 20 to 300 nm, and the primary micro-emulsion can be optionally converted to a secondary W/O micro-emulsion or an O/W micro-emulsion, by means of reaction with an aqueous phase.

In particular, in a preferred embodiment, the aforementioned emulsions have 0.01 to 15 wt.-% of one or more alcohols d) as described.

It is furthermore preferred if the micro-emulsion contains 0 to 30 wt.-%, particularly 0.01 to 30 wt.-% of one or more water-soluble or fat-soluble active substances, or mixtures of water-soluble and fat-soluble active substances.

Particularly preferably, both water-soluble and fat-soluble active substances are contained.

It is also advantageous if, in addition, 0 to 15 wt.-%, preferably 0.01 to 15 wt.-% additives are present, which are particularly selected from among diffusion reinforcing agents, penetration reinforcing agents, chelation agents, electrolytes, oxidants, moisturizers, bleaches, preservatives, or mixtures thereof. Here, electrolytes, diffusion reinforcing agents, oxidants, chelating substances, penetration promotion agents, moisturizers, or mixtures thereof are particularly preferred.

Particularly preferably, the micro-emulsion according to the invention has one or more oxygen carriers. The latter is/are particularly selected from among hemoglobin, myoglobin, and mixtures thereof, whereby hemoglobin is preferred. The oxygen carrier(s) can be present in amounts of 0.001 to 20 wt.-%, particularly 0.01 to 15 wt.-%, especially 0.1 to 10 wt.-%. For this purpose, in another preferred embodiment, the water-soluble and/or fat-soluble active substances listed below can then be contained. For this purpose, one or more of the stated additives can also be contained in the amounts indicated, in another embodiment.

Another preferred embodiment is represented by micro-emulsions that contain
50 to 80 wt.-% of an oil phase,
5 to 40 wt.-% of a mixture of one or more W/O and one or more O/W surfactants in the ratio indicated above;
0.1 to 10 wt.-% of one or more lecithins, phosphatidyl cholines, or derivatives or mixtures thereof as an emulsifier;
0.00 to 10 wt.-%, particularly 0.1 to 10 wt.-%, of one or more monovalent or bivalent C₁₋₈ alcohols;
1 to 10 wt.-% water or aqueous solutions.

In this connection, active substances of the type indicated can be present in the amount indicated, as described.

The amount of oils is 45 to 90, particularly 45 to 80, and preferably 45 to 60 wt.-%. In particular, the oil phase consists of liquid oils, such as the esters of alkane carboxylic acids, for example. These are preferably selected from among isopropyl myristate, isopropyl palmitate, isopropyl oleate, isooctyl stearate, isononyl stearate, and the like. Furthermore, dialkyl ethers, fatty alcohols having 6-18 carbon atoms, or triglycerine esters of saturated and/or unsaturated alkane carboxylic acids are preferred. These particularly include synthetic, semi-synthetic, and natural oils, such as olive oil, almond oil, avocado oil, sunflower oil, soybean oil, peanut oil, canola oil, and the like.

Any desired mixtures of such oils and ester oils can preferably be used within the sense of the present invention.

The ratio of W/O to O/W surfactant or mixtures of the surfactants is preferably 1:4 to 1:1.2, preferably 1:3 to 1:1.2, and particularly 1:2 to 1:1.3.

Particularly preferred W/O and O/W surfactants are non-ionic and have an HLB value of 3 to 7 or 9 to 18, respectively. Preferably, they are selected from among the group of sorbitan ethers, of the ethoxylated sorbitan derivatives. Furthermore, the surfactants can advantageously be selected from among the group of ethoxylated fatty alcohols having 8-18 carbon atoms in straight chains, of glyceryl ethers/esters of saturated and unsaturated fatty acids, ethoxylated glyceryl esters, preferably diglycerides and triglycerides, of ethoxylated alkyl ethers, or of fatty alcohol (C16-C18) glucosides or suitable mixtures of the surfactants, in each instance.

The amount of non-ionic surfactants (one or more compounds) in the preparations amounts to 0.1 to 45 wt.-%, preferably 1-45 wt.-%, particularly 5-40 wt.-%, or even 5 to 35 wt.-%, with reference to the total weight of the preparation.

The emulsifier is particularly selected from among lecithin or phosphatidyl cholines or derivatives or mixtures thereof, such as lecithin from plants (soybean, canola, cottonseed) and egg yolk; phosphatidyl choline from soybean and egg yolk; mixtures of phosphatidyl choline and lecithin in various ratios such as NAT products, phosphatidyl ethanol amine; phosphatidyl serine; phosphatidyl inosite from soybean, canola, cottonseed; hydroxylated lecithin.

The amount of emulsifier(s) in the compositions amounts to 0.01 to 20, preferably 15 wt.-%, especially 0.1 to 10 wt.-%, preferably 0.5-5 wt.-%, particularly 1-5 wt.-% with reference to the total weight of the composition.

The alcohol(s) is/are preferably selected from among univalent C₁₋₈ alcohols such as ethanol, propanol, isopropanol; or from among bivalent alcohols such as glycols, e.g. propylene glycol, 1,2-octane diol, 1,2-hexane diol.

The amount of alcohol(s) in the compositions amounts to 0.0 to 15, especially 0.01 to 15, and particularly 0.1 to 15 wt.-%, preferably 1 to 15 wt.-%, particularly preferably 5-15 wt.-%, with reference to the total weight of the composition.

Water-soluble active substances that are preferably selected are amino acids, peptides, protein hydrolysates, proteins, saccharides, oligosaccharides, polysaccharides, and derivatives thereof, hormones and substances similar to hormones, antioxidants, vitamins and pro-vitamins, AHA acids, moisturizers such as NMF, oxidants, plant extracts, flavonoids, and plant polyphenols or mixtures thereof.

Fat-soluble active substances that are preferably selected are antioxidants, vitamins, pro-vitamins, unsaturated fatty acids, ceramides or mixtures thereof. If necessary, prostaglandins, but particularly agents having a dermatological effect, selected from among hormones and substances similar to hormones, antimycotics, keratinolytics, keratinoplastics, scar treatment agents, tanning agents, tars, acelainic acid, photocumarins, or mixtures thereof also can be.

In a particularly preferred embodiment, the micro-emulsion according to the invention contains proteins selected from among native, modified and/or unmodified hemoglobin, myoglobin, or mixtures thereof, particularly not modified, in a total amount of 0.001 to 20 wt.-%, particularly 0.1 to 20 wt.-%, or also 0.1 to 20 wt.-%, particularly 1 to 15 wt.-%, particularly 1 to 15 wt.-%, especially 1 to 10 wt.-%.

In this connection, it is preferred if, in addition, antioxidants, protein stabilizers, monosaccharides, oligosaccharides, and polysaccharides, particularly glucose, collagen, moisturizers, amino acids, or mixtures thereof are furthermore contained as additives. In particular, especially in the case of products containing hemoglobin/myoglobin, antioxidants, glutathione, super-oxide dismutase, melatonin, flavonoids, amino acids, if necessary furthermore also collagen, glucose, amino acids and moisturizers, or mixtures thereof are present.

A very particularly preferred micro-emulsion according to the invention comprises unmodified hemoglobin, myoglobin, or mixtures thereof in an amount of 1 to 10 wt.-%, as well as 1 to 5 wt.-% glucose, 0.01 to 5 wt.-% amino acids natural for humans, as active substances.

In this connection, emulsions that contain not only the aqueous phase but also 50 to 90 wt.-% of a liquid oil phase, with oils selected from among isopropyl myristate, isopropyl palmitate, isopropyl oleate, isooctyl stearate, isononyl stearate, are especially preferred. Furthermore, dialkyl ethers, fatty alcohols having 6-18 carbon atoms, or triglycerin esters or saturated and/or unsaturated alkane carboxylic acids are preferred. Among these, synthetic, semi-synthetic, and natural oils, such as olive oil, almond oil, avocado oil, sunflower oil, soybean oil, peanut oil, canola oil, as well as 0.1 to 40 wt.-%, especially 0.1 to 30, or also 0.1 to 20 wt.-% of a surfactant mixture, selected from among the group of the sorbitan ethers, the ethoxylated sorbitan derivatives, are particularly suitable. Furthermore, the surfactants can advantageously be selected from among the group of ethoxylated fatty alcohols having 8-18 carbon atoms in straight chains, of glyceryl ethers of saturated and unsaturated fatty acids, ethoxylated glyceryl esters, of ethoxylated alkyl ethers, or of fatty alcohol (C16-C18) glucosides or suitable mixtures of the surfactants, in each instance, having an HLB value <8, as W/O surfactants and, as O/W surfactants, those from the group of sorbitan ethers, of ethoxylated sorbitan derivatives. Furthermore, these surfactants can advantageously be selected from among the group of ethoxylated fatty alcohols having 8-18 carbon atoms in straight chains, of glyceryl ethers of saturated and unsaturated fatty acids, ethoxylated glyceryl esters, of ethoxylated alkyl ethers, or of fatty alcohol (C16-C18) glucosides or suitable mixtures of the surfactants, in each instance, having an HLB value >10, particularly together with 0.1 to 8 wt.-% phosphatidyl choline or products containing phosphatidyl choline, such as NAT-8539 (Rhone-Poulenc), as well as 0.1 to 5 wt.-% ethanol, isopropanol, 1,2-octane diol, or mixtures thereof.

It can also be advantageous if the aforementioned micro-emulsions additionally contain water-soluble and/or fat-soluble vitamins/pro-vitamins in an amount of 0.01-1.0 wt.-%.

In another preferred embodiment, the emulsion according to the invention contains plant extracts in an amount of 0.1 wt.-% to 5, particularly up to 3.0 wt.-%, 0.1 to 5.0 wt.-% ether oils, 0.1 to 10 wt.-% AHA acids, 0.01 to 3 wt.-% Hormones or substances similar to hormones, 0.1 to 5 wt.-% essential fatty acids, ceramides (0.1 to 5 wt.-%) or mixtures thereof.

As mentioned above, the micro-emulsion according to the invention can be applied topically, or a mixture thereof with 1 to 90, preferably 10 to 90 wt.-% of an aqueous phase is prepared, whereby the preparation then has 1 to 50 or even 5 to 50, particularly 1 to 40 wt.-% of the oil phase, and represents a W/O or an O/W micro-emulsion.

These primary micro-emulsions can particularly absorb 1 to 30% water or aqueous solutions, and thereby a secondary W/O micro-emulsion having substances enclosed in water droplets is formed. The droplets have a diameter between 50 and 400 mm.

By means of dilution with water or aqueous solutions, the primary micro-emulsions can convert to O/W micro-emulsions that have oil droplets (1-40%) having a diameter of 40 to 300 nm, in a continuous aqueous phase (60-95%).

Such a mixture can, in particular, have 26 to 50 wt.-% oil phase, and represent a secondary W/O micro-emulsion, or can have 5 to 25 wt.-% oil phase and then represent a secondary O/W micro-emulsion.

The micro-emulsions according to the invention are produced in that the oil phase, containing the surfactant(s), the emulsifier(s), as well as the alcohol(s) and, if applicable, the water-soluble substances contained therein, and an aqueous phase, and, if applicable, the water-soluble active substance(s), are mixed with one another at temperatures from 10 to 30° C., and the primary W/O micro-emulsion obtained in this manner is converted to a secondary W/O micro-emulsion or a secondary O/W micro-emulsion, if necessary with an aqueous phase that can contain additional water-soluble active substances, if applicable.

The micro-emulsions according to the invention are particularly suitable as cosmetic, dermatological, pharmaceutical preparations for the topical treatment of skin that has been irritated or damaged or has degenerated due to allergy, bacteria, immunology, external influences, or for the care or treatment of hair, such as for cleaning and conditioning, the latter in the case of dry or damaged hair, and hair that is difficult to comb.

Very particularly, they can be used for the treatment of skin that has been irritated or changed due to age. For this purpose, the preparations containing hemoglobin, myoglobin, or mixtures thereof, advantageously containing the other active substances, additives, such as hydrogen peroxide, in particular, are especially suitable.

The treatment of neurodermatitis, acne, and psoriasis are also particularly possible. Also, the agent according to the invention can be used against inflammations, also when using the oxygen carriers.

It has been shown that by means of the emulsion according to the invention, also the stated active substances penetrate as far as the vital cells of the stratum germinativum, particularly the aforementioned biological oxygen carriers, glucose and, if applicable, the other active substances and/or additives that are present, such as moisturizers, vitamins, essential fatty acids and lipids, trace elements, antioxidants, amino acids, furthermore also peptides, monosaccharides, oligosaccharides and polysaccharides, oligonucleotides, ancillary substances.

The primary and secondary W/O micro-emulsion according to the invention therefore, in a particularly preferred embodiment, has a continuous oil phase that contains droplets of the discontinuous aqueous phase, which can essentially contain the following components:
one or more biological oxygen carriers, if applicable in combination with one or more protein stabilizers; one or more antioxidants;
one or more vitamins and pro-vitamins; one or more monosaccharides, oligosaccharides and polysaccharides and their derivatives; one or more amino acids, peptides, and protein hydrolysates; one or more proteins and protein derivatives; one or more hormones and substances similar to hormones; one or more plant extracts;
one or more diffusion reinforcing agents; one or more penetration-promoting agents; one or more inorganic salts; one or more chelate-forming agents; one or more oxidizing substances (H₂O₂, hydroquinone); one or more chemical oxygen carriers; one or more water-soluble active substances (pharmaceuticals, dermatological substances).

The oil phase particularly contains the surfactants, the emulsifier(s) and alcohol(s), antioxidant(s), vitamin(s) and pro-vitamin(s), essential fatty acids, ceramides, prostaglandins, ether oils, lipophilic active substances from the group of the pharmaceuticals, dermatological substances.

In this connection, the individual ingredients are present in the amounts indicated above, particularly the preferred proportions.

Surprisingly, it was found that hemoglobin or myoglobin bound into the micro-emulsion according to the invention can penetrate quickly and deeply into the stratum corneum, and distributes homogeneously there (see use examples). With this, a diffusion of oxygen that is facilitated for hemoglobin introduced into the stratum corneum is achieved.

For the optimal effect of the facilitated diffusion, oxygen affinity and oxygen cooperativity of the oxygen binder (hemoglobin, myoglobin) must be adjusted advantageously. A measure for the former is the semi-saturation pressure (P₅₀), and for the latter it is the HILL index. The cooperativity must be as great as possible, for whole blood its value is about 2.6.

The affinity, on the one hand, must be so great (i.e. the P₅₀ value must be so small) that the oxygen from the air is still absorbed well, but on the other hand, the affinity must be so low that the oxygen that is absorbed can also easily be issued back to the cells of the skin epithelium. Hemoglobin has an antioxidant effect in two ways, according to the invention, namely, as explained, by means of a reduction in the oxygen tension and by means of its catalase effect. A third effect is inhibition of inflammation and a fourth is photo-protection.

With regard to the biological oxygen carrier, it is advantageous, particularly in the case of hemoglobin, to chemically modify the affinity, but this can also be done by means of non-covalently bonded effectors that are mixed into the preparation. Chemical modification can take place, for example, with pyridoxal phosphate. Covalent modification can take place, for example, with 2,3-diphosphoglycerate or artificial effectors such as inositol hexaphosphate or mellitic acid, in 1-3 times, particularly an approximately equivalent amount, with reference to hemoglobin and/or hemoglobin/myoglobin.

If necessary, further stabilization of the biological oxygen carrier with regard to the function structure of the proteins can take place, because the surfactants present in the emulsion can also influence the function of oxygen binding of the hemoglobin.

Human or bovine hemoglobin, but preferably porcine hemoglobin, stabilized with carbon monoxide (CO), is particularly preferred. The production of such a stabilized hemoglobin is described in DE 1 970 103.7 (corresponding to U.S. Pat. No. 5,985,332). According to this reference, hemoglobin/myoglobin can be completely transformed to carboxyhemoglobin/myoglobin, which is stable in storage and does not need to be de-ligandized before further use, by means of equilibration with carbon monoxide. Carbonylation is also possible with modified hemoglobin.

The activation of the oxygen carrier can then by means of local gasification with oxygen of the skin to which the emulsion was applied.

The hemoglobin can, as mentioned, be present in a mixture with myoglobin, particularly the latter in amounts of 0.1 to 50%, with reference to the hemoglobin amount. Preferably, myoglobin is used with hemoglobin in amounts of 50 to 70% hemoglobin and 50-30% myoglobin, particularly 75 to 90% hemoglobin and 25 to 10% myoglobin. In this connection, the % information relates to proportions by mass.

Human hemoglobin, porcine hemoglobin, which is preferred, or bovine hemoglobin can particularly be used as hemoglobin. The type of myoglobin is also optional, it can be obtained from different animal species, e.g. from dogs, sheep, horses, or whales.

Non-modified native hemoglobin and/or myoglobin is particularly preferably used, which can be particularly preferably protected against oxidation by means of carbonylation, whereby the oxygen carrier solution had a non-chemically reactive effector, as mentioned, particularly 2,3-diphosphoglycerate, in a 1 to 3 times, preferably equivalent amount with reference to the hemoglobin/hemoglobin/myoglobin. Furthermore, in addition or alternatively, hemoglobin chemically modified with pyridoxal effectors can also be used. For this purpose, hemoglobin is converted with the corresponding effectors mentioned, if necessary carbonylated.

Very preferably, non-modified human hemoglobin or particularly porcine hemoglobin de-oxygenated according to the invention, if necessary carbonylated, and non-modified myoglobin of dogs, sheep, or horses, which has been correspondingly de-oxygenated, are used.

NaCl, KCl, and NaHCO₃, can be present as suitable electrolytes together with the oxygen carrier(s), particularly in physiological amounts (in mM): NaCl 125; KCl 4.5; NaHCO₃ 20). Alternatively, hemoglobin or myoglobin can also be linked with polyalkylene oxides for stabilization, as a modification, as described in the patents U.S. Pat. No. B 4,179,337, U.S. Pat. No. B 5,478,805, U.S. Pat. No. B 5,386,014, U.S. Pat. No. B 5,312,808, or EP 0 206 448, EP 067 029. Furthermore, the oxygen carrier can also be polymerized or polymerized and pegylated, converted with effectors, and/or carbonylated, as described in DE A 100 31 740 (WO 02/00230), DE A 100 31 744, DE A 100 31 742. The content of these references is therefore incorporated.

Surprisingly, it was found that a micro-emulsion containing hemoglobin or myoglobin can be produced directly before its use. Amazingly, different skin tones can be achieved in this connection (see use example).

For the cosmetic or dermatological treatment of normal, aged, and degenerated skin, primary micro-emulsions according to the present invention can be produced in the form of micro-dispersed water droplets (0.1-40%) having a diameter of 30 to 400 nm, in a continuous oil phase (30-70%) that contains surfactants and alcohol/emulsifier (1-40%).

These primary micro-emulsions can absorb from 1 to 30% water or aqueous solutions, and a secondary W/O micro-emulsion having substances enclosed in water droplets forms as a result. The droplets have a diameter between 50 and 400 mm.

By means of dilution with water or aqueous solutions, the primary micro-emulsions can convert to O/W micro-emulsions, which have oil droplets (1-40%) having a diameter from 40 to 300 nm, in a continuous aqueous phase (60-95%).

Both the primary and the secondary emulsions can penetrate into the stratum corneum of the skin very quickly and deeply, whereby W/O micro-emulsions are present in liquid form to liquid gels or gels, and the O/W micro-emulsions are present in liquid and sprayable form.

In this connection, there is no systemic effect, as it was possible to determine using an insulin test. A micro-emulsion according to the following Example 1, Table 1, Product 1, containing insulin in usual amounts, was applied to 2 test subjects. A determination of the blood glucose level after three hours did not show any decrease.

In the following, the individual components of the micro-emulsion according to the invention will be described in greater detail:

1. Oils

The oil phases of the micro-emulsions according to the invention are advantageously selected from among the following substances:
a.) Esters from C₉₋₁₈ alkane carboxylic acids and C₃₋₃₀ alcohols, such as, in particular, isopropyl myristate, isopropyl palmitate, isopropyl stearate, isopropyl oleate, n-butyl stearate, n-hexyl laurate, n-decyl oleate, isooctyl stearate, isononyl stearate, isononyl isonanoate, 2-ethyl hexyl palmitate, 2-ethyl hexyl laurate, 2-hexyl decyl stearate, 2-octyl dodecyl palmitate, ethyl oleate, oleyl oleate, oleyl ecurate, erucyl oleate, as well as synthetic, semi-synthetic, and natural mixtures of such esters. Preferably, the oil phase is selected from among pharmaceutically compatible oils such as isopropyl myristate, ethyl oleate, isopropyl palmitate, isopropyl oleate, oleyl oleate, isooctyl stearate.
b.) Saturated, unsaturated, long-chain fatty acids of animal and plant origin, particularly oleic acid, palmitinic acid, or oleic acid, or folic acid and its derivatives, extracts or other products of plant or animal origin, e.g. evening primrose oil, borage oil, or red currant seed oil, Particularly are also evening primrose oil, borage oil, γ-linolenic acid;
c.) Dialkyl ethers of the group of alcohols, as well as of fatty acid triglycerides, particularly triglycerin esters of saturated and/or unsaturated alkane carboxylic acids, having a chain length of 8 to 24, particularly 12 to 18 C atoms. Particularly preferred are synthetic, semi-synthetic, and natural oils, e.g. olive oil, almond oil, avocado oil, sunflower oil, soybean oil, peanut oil, canola oil, palm oil, coconut oil, palm kernel oil, and the like. Furthermore, the pharmaceutically compatible oils 2-ethyl hexyl isostearate, octyl dodecanol, isotridecyl isonanoate, isoeicosan, 2-ethyl hexyl cocoate, capryl caprinic acid triglyceride, dicaprylyl ether, are preferred.
d.) Hydrocarbons with low volatility, particularly paraffin oil, squalene, and squalane.
e.) Fatty alcohols having 6-18 carbon atoms in straight chains such as lauryl alcohol, palmitinic alcohol, myristinic alcohol, araquidone, linolenic alcohol and linolic alcohol.

Particularly preferred oils are, along with the ones already mentioned, also isopropyl myristate, isopropyl palmitate, isopropyl stearate, isopropyl oleate, olive oil, almond oil, avocado oil, sunflower oil, soybean oil, peanut oil, canola oil, capryl caprinic acid triglyceride, dicaprylyl ether, squalane, or mixtures thereof.

Furthermore, mixtures of individual oils as well as of individual groups, as well as of different groups are also possible.

2. O/W and W/O Surfactants

The surfactants are preferably selected from among non-ionic substances. There are both O/W surfactants and W/O surfactants from these groups. The former are characterized, among other things, by an HLB value ≧8, the latter usually have an HLB value of ≦8. Surfactants having an HLB value from 2 to 6 together with those having an HLB value from 12 to 20 are preferably used.
a.) Sorbitan derivatives, particularly sorbitan monolaurate and sorbitan trioleate.
b.) Ethoxylated sorbitan derivatives, such as, in particular, polyethylene glycol (20) sorbitan monolaurate, polyethylene glycol (20) sorbitan monostearate, polyethylene glycol (20) sorbitan mono-oleate.
c.) Glyceryl ethers of saturated and unsaturated fatty acids such as monoglycerin, diglycerin, triglycerin, and polyglyceryl derivatives, including polyglyceryl diisostearate, polyglyceryl-2-oleyl ether, polyglyceryl-6-distearate, polyglyceryl-4-oleyl ether.
d.) Ethoxylated glyceryl esters such as ethoxylated triglycerides, e.g. polyethylene glycol (20) glyceryl tristearate;
   Among the glycerides, diglyceride and triglyceride/derivatives thereof are preferred.
f.) Ethoxylated alkyl ethers, particularly polyethylene glycol dodecyl ether (Brij30), polyethylene glycol hexadecyl ether (Brij52).
g.) Fatty alcohol (C16-C18) glucosides such as sucrose stearate, sucrose palmitate, Plantacare 1200 UP and Plantacare 2000 UP
h.) Ethoxylated fatty alcohols with 8-18 carbon atoms in straight chains, particularly (polyethylene glycol (2) stearyl ether (Steareth-2), from Steareth-13 to Steareth-20, from Oleth-3 to Oleth-15, from Cetech-13 to Cetech-20, from Ceteareth-12 to Ceteareth-30 (INCI).

The amount of the non-ionic surfactants (one or more compounds) in the preparations is preferably 10 to 50 wt.-%, particularly preferably 10-55 wt.-%, particularly 10-40 wt.-%, with reference to the total weight of the preparation.

3. Emulsifiers

a). Phospholipids, particularly lecithin from plants (soybean, canola, cottonseed) and egg yolk; phosphatidyl choline from soybean and egg yolk or mixtures thereof such as NAT 8539); phosphatidyl ethanol amine; phosphatidyl serin, phosphatidyl inosite from soybean, canola, cottonseed, hydroxylated lecithin, mixtures of phosphatidyl choline and lecithin in variable proportions, such as NAT 8539 or the like, for example.
   Particularly preferred are lecithin from soybean and egg yolk, e.g. under the trade names Epikuron 135, Epikuron 170, Epikuron 200, Epikuron 200 SH (Lukas Meyer), Phospholipon 25, NAT-8539 (Nattermann).
b.) Cholesterol and cholesterol derivatives, such as, for example, ethoxylated cholesterol such as polyethylene glycol (10) soy ester oil, are used.
   4. Alcohols
a.) Monovalent C₁₋₈ alcohols such as ethanol, propanol, isopropanol, butanol;
b.) Glycols such as propylene glycol, 1,2-octane diol, 1,2-hexane diol;
   Ethanol, propanol, 1,2-octane diol, propylene glycol are particularly preferred.
      5. Active Substances
      1. Active Substances Soluble in Fat
a.) Fat-soluble vitamins such as Vitamin A and its derivatives, Vitamin C and its derivatives, Vitamin E and its derivatives. Tocopherol acetate, ascorbic acid palmitate are particularly preferred.
b.) Antioxidants such as carotinoids, carotine (e.g. α-carotine and β-carotine and their derivatives), α-tocopherol and its derivatives, ascorbic acid palmitate.
c.) Ether oils, particularly terpenes (monoterpenes, sesquiterpenes, diterpenes): citrus oil, Italian pine, chamomile; alcohols (monoterpenols, sesquiterpenols, diterpenols): ravensara, hyssop, niaouli; aldehydes: melissa, eucalyptus; ketones: (monoterpene ketones, sesquiterpene and diterpene ketones): yarrow, thuja; as well as esters (monoterpene esters): lavender oil, ylang ylang; phenols: thyme; phenyl ethers: anise, clove; oxides: (cenol); lactones: patchouli, incense, and cumarins.
   Very particularly preferred are clove oil, thyme oil, mint oil, citrus oil, Italian pine, lavender oil, ylang ylang, chamomile, ravensara, hyssop, niaouli, anise, patchouli, incense, yarrow, thuja, birch oil, melissa, eucalyptus. Furthermore, the ether oils are preferably selected from among citrus oil, Italian pine, chamomile, ravensara, hyssop, niaouli, melissa, eucalyptus, yarrow, thuja, lavender oil, ylang ylang, Melaleuca.
   The amount of the ether oils (one or more compounds) in the preparations is preferably 0.1 to 10 wt.-%, particularly preferably 0.5 to 5 wt.-%, particularly 1-5 wt.-% with reference to the total weight of the preparation.
d.) Essential fatty acids, particular linolic and γ-linolenic acid, or essentially oils containing fatty acids. Particularly preferably γ-linolenic acid, borage oil, evening primrose oil.
e.) Ceramides such as ceramide I, ceramide II.
   2. Active Substances Soluble in Water
a.) Antioxidants such as hydroxy benzoates and dihydroxy benzoates, hippurates, salicylates, cysteine and derivatives thereof, glutathione, Vitamin C and its derivatives (e.g. Mg-ascorbyl phosphate, ascorbyl acetate), Vitamin H, super-oxide dismutase, catalase; amino acids (e.g. glycine, histidine, thryonine) and their derivatives, imidazoles, thiolene, (glutathione, thioredoxyn, cysteine, cystamine, and their derivatives), flavonoids, melatonin, Vitamin E and its derivatives.
   Ascorbyl acetate, super-oxide dismutase, cysteine, and glutathione are particularly preferred.
b.) Vitamins and pro-vitamins such as Vitamin B complex, Vitamin C and derivatives, Vitamin H and derivatives, biotin, pantothenic acid, pantenol.
c.) Saccharides and oligosaccharides such as glucose, fructose, mannose, mannitol, inosite, N-acetyl-D-glucosamine, D-glucosamine, chito-oligosaccharides, raffinose, trehalose. Saccharides and oligosaccharides, particularly glucose, D-glucosamine, N-acetyl-D-glucosamine, chito-oligosaccharides, trehalose are particularly preferred.
d.) Polysaccharides such as chitosan, hyaluronic acid, heparin, dextran, cellulose ester, alginic acid.
   Here, chitosan, hyaluronic acid are particularly preferred
e.) Proteins and protein derivatives such as hemoglobin, myoglobin, collagen, fibrin, elastin. Here, in addition to the oxygen carriers, collagen is particularly preferred.
f.) Hormones and substances similar to hormones such as hydrocortisone and its derivatives, melatonin, glycyrrhitinic acid and its derivatives, as well as other plant hormones. Here, melatonin, glycyrrhitinic acid and derivatives are particularly preferred.
g.) Plant extracts (extracts themselves or other products having a plant or animal origin) are preferably selected from among meristern extract, aloe vera, echinacea, hammamelis extract, asparagus extract, cashew tree, horse chestnut, arnica, calendula.
   Meristern extract, aloe vera, Echinacea, ivy, nettle, chamomile, horsetail are particularly preferred.
h.) Amino acids, peptides, and protein hydrolysates, such as all natural amino acids suitable for mammals, particularly humans, protein hydrolysates, e.g. silk protein hydrolysate, yeast hydrolysate, wheat protein hydrolysates. Here, the natural amino acids, peptides and protein hydrolysates such as silk protein hydrolysate, yeast hydrolysate are particularly preferred.
i.) Chemical oxygen carriers, such as organic and inorganic peroxides, e.g. hydrogen peroxide, benzoyl peroxide. Hydrogen peroxide is preferred.
k.) Moisture-retaining substances such as the so-called NMF (actors), particularly glycerin, ectoins, sorbitol, pyrrolidone carboxylic acid, urea, allantoin, glucosamine, trehalose, chito-oligosaccharides, carboxylic acid, hydroxy carboxylic acid and dicarboxylic acid, as well as polysaccharides—hyaluronic acid, chitosan, aloe vera extract. Glycerin, urea, sorbitol, allantoin, pyrrolidone carboxylic acid, lactic acid, hyaluronic acid, chitosan, aloe vera extract, chito-oligosaccharides are preferably selected.
   The amount of such active substances (one or more compounds) in the preparation is preferably 0.01 to 15 wt.-%, particularly preferably 0.5-10 wt.-%, particularly 1-5 wt.-%, with reference to the total weight of the composition.
      6. Additives

The micro-emulsions according to the invention can also have one or more of the following additives. In this connection, it has been shown that electrolytes, for example, do not exert any influence on the emulsion, as reported in the state of the art, in other words they do not influence the hydrophilia-lipophilia balance of the surfactants.
a.) Electrolytes, particularly of one or more salts with the following anions: chloride, sulfate, carbonate, phosphate. Electrolytes based on organic anions can also be used to advantage, for example lactates, acetates, benzoates, salicylates, propionates, tartrates, citrates, and others. Ammonium ions, alkyl ammonium ions, alkali metal ions, earth alkali metal ions, magnesium ions, iron ions, and zinc ions are preferably used as cations of the salts. Potassium chloride, cooking salt, magnesium sulfate, zinc sulfate, and mixtures thereof are particularly preferred. Salt mixtures such as those that occur in the natural salt of the Dead Sea, in amounts of 0.1 to 2.0%, are also advantageous.
b.) Chelating substances (ethylene diamine tetra-acetic acid and its salts, deferoxamine, histidine). Here, ethylene diamine tetra-acetic acid is preferred.
c.) Chemical and natural bleaches, such as hydroquinone, Kojak acid, arbutin, acelainic acid, lemon juice and cucumber juice. Here, arbutin, acelainic acid, or also hydroquinone are particularly preferred.
d.) Chemical or natural tan-producing agents, such as walnut shell extract, alloxan, 1,3-dihydroxy propan-2-one.
e.) Preservatives such as salicylates, benzoates, parabens, whereby salicylic acid and phenyl ethanol are particularly preferred.
f.) Diffusion reinforcing agents such as menthol and others from the group of pinene, thymol, camphor, caffeine, diethylene glycol ethers, e.g. diethylene glycol monoethyl ether, diethylene glycol, cineol, menthol, propylene glycol, butylene glycol, polyethylene glycol from 4 to 250 ethylene glycol groups, diethylene glycol ester, e.g. diethylene glycol monoethyl ester, oleic acid, salicylic acid, α-hydroxy acids.
   Advantageously, the diffusion reinforcing agents are selected from the group of menthol and diethylene glycol monoethyl ether.
g.) Penetration reinforcing agents such as urea, ethanol, dimethyl silfoxide, cysteine.

The additives are present first in the aqueous phase or in the oil phase, depending on the chemical consistency.

The amount of additives is preferably 0.1 wt.-% to 10 wt.-%.

It is possible and advantageous, if applicable, to use the preparations according to the invention as the basis for pharmaceutical formulations. The transitions between pure cosmetics and pure pharmaceuticals are not rigidly defined, in this connection. According to the invention, fundamentally all classes of active substances, such as water-soluble and fat-soluble, are suitable as pharmaceutically active substances, whereby lipophilic active substances are preferred. Examples of this are: antihistaminics, antiphlogistics, antibiotics, antimycotics, virostatics, active substances that promote blood circulation, keratolytics, keratoplastics, hormones, steroids, vitamins, and others.

It is advantageous to add additional anti-irritative or anti-inflammatory active substances to the preparations in the sense of the present invention, particularly bisabolol and/or panthenol, glycyrrhitinic acid and its derivatives, hydrocortisone-17-valerate and its derivatives.

Medical topical compositions in the sense of the present invention generally contain one or more medications in an effective concentration.

### EXAMPLES

The invention will be explained in greater detail using the following examples.

The emulsions according to the invention are produced in that the surfactants, alcohol, if present, emulsifier, additives, if applicable, are mixed and combined with the oil phase at room temperature, while stirring. Subsequently, at the same temperature, while stirring, the aqueous phase, containing water-soluble active substances and/or additives, if applicable, is added and stirred until a clear solution was obtained. The particle (micelle) size is 50 to 400, particularly 20 to 300 nm. All compounds or components that can be used in the cosmetic formulations are either known and commercially available or can be synthesized according to known methods. All of the % information is weight percent (w/w) unless otherwise indicated.

### Example 1

### Primary W/O Type Micro-Emulsion

TABLE 1
(information in wt. -%)

| | 1 | 2 | 3 | 4 | 5 | 6 |
|---|---|---|---|---|---|---|
| Water | 10.0 | 5.0 | 10 | 10.0 | 10.0 | 9.7 |
| Tween 80 (O/W) | 22.7 | 22.7 | 26.0 | 25.0 | 25.0 | 22.0 |
| Polyglycerin-2-oleate | | | | | 10 | |
| Span 80 | 11.4 | 11.4 | 8.0 | 9.0 | | 11.0 |
| Ethanol, 96% | 4.5 | | | | | 3.0 |
| Phosphatidyl choline | 1.4 | 1.5 | | | | |
| 1,8-octane diol | | | | | 10.0 | |
| NAT-8359 (Rhone-Poulenc) | | | 2.0 | 3.0 | | 2.0 |
| Propanol | | 5.0 | | 3.0 | | |
| Ethyl oleate | | 50.4 | | | | |
| Isopropyl myristate (IPM) | 50.0 | | 50.0 | | | 52.0 |
| Myritol 318 | | | | 45.0 | 45.0 | |
| Diethylene glycol monoethyl | | 4.0 | 4.0 | | | |
| ether | | | | | | |
| DMSO | | | | 5.0 | | |
| Hydrogen peroxide, 33% | | | | | | 0.3 |

### Example 2

### Primary W/O Micro-Emulsion With Natural Oils or Mixtures Thereof

TABLE 2

| | 1 | 2 | 3 | 4 | 5 |
|---|---|---|---|---|---|
| Water | 10.0 | 5.0 | 10.0 | 10.0 | 10.0 |
| Almond oil/IPM (30/70) | 49.0 | | | | |
| Olive oil/ethyl oleate (30/70) | | 52.6 | | | |
| Jojoba oil/myritol (30/70) | | | 49.0 | | |
| Almond oil/avocado oil/IPM | | | | 52.0 | 41.0 |
| (15/15/70) | | | | | |
| Tween 80 | 23.0 | 22.0 | 25.0 | 22.0 | 33.0 |
| Span 80 | 11.5 | 11.4 | 8.0 | 9.0 | 11.0 |
| Ethanol, 96% | 5.0 | 5.0 | 5.0 | 5.0 | 5.0 |
| Phosphatidyl choline | 1.5 | | | 2.0 | |
| NAT-8539 (Rhone-Poulenc) | | 4.0 | 3.0 | | |

### Example 3

### Primary W/O Micro-Emulsion With Ether Oils

The W/O micro-emulsions were produced as in Example 1, Formulation No. 1 (Table 1) and Formulation No. 2 (Table 2), whereby instead of IPM, oil mixtures of ether oils (10%) in IPM, ethyl oleate, or mixtures thereof with natural oils were used. The following ether oils, separately or as mixtures in any desired ratios, were used:
Citrus oil, Italian pine, lavender oil, ylang ylang, chamomile, ravensara, hyssop, niaouli, anise, clove, thyme, patchouli, incense, yarrow, thuja, melissa, eucalyptus, rosa rubignosa, innophylum callophylum, as well as 1,4-cenol, menthol, and limes.

### Example 4

### Secondary O/W Micro-Emulsion With Ether Oils

Part of the primary W/O micro-emulsion containing ether oils, from Example 3, was mixed with 5 parts water or aqueous solutions or hemoglobin solutions. This resulted in a clear, opaque O/W micro-emulsion having a particle size of 100 to 200 nm.

### Example 5

### Primary W/O Micro-Emulsion With Unsaturated Fatty Acids

The W/O micro-emulsions were produces as in Example 1 No. 1 (Table 1) and No. 1 (Table 2), whereby instead of IPM, oil mixtures of unsaturated fatty acids (10%) in IPM or ethyl oleate were used. The following unsaturated fatty acids, separately or as mixtures in any desired ratios, were used, namely linolic and linolenic acid, borage oil, and rosehip oil.

### Example 6

### W/O Micro-Emulsion With Fat-Soluble Vitamins and Pro-Vitamins

The following vitamins were dissolved separately or as mixtures, in an oil, or IPM, myritol, ethyl oleate, in concentrations of 0.01 to 10%. The primary W/O micro-emulsions were mixed by adding the surfactants/emulsifier/alcohol solution and subsequently water or aqueous solutions, as described in Example 1 (No. 1 to 5). The particle size of the W/O micro-emulsion lies in the range of 50 to 100 nm.

### Example 7

### O/W Micro-Emulsion With Fat-Soluble Vitamins and Pro-Vitamins

Part of the primary micro-emulsion according to Example 5 was mixed with 5 parts water or aqueous solutions. This resulted in a clear, opaque O/W micro-emulsion solution having a particle size of 100-200 nm.

### Example 8

### W/O Micro-Emulsion Containing Hemoglobin

Hemoglobin preparation: antioxidants: L-cysteine and N-AC-cysteine, (0.1-0.03%), glucose (1.0%), preservative: salicylate NA (K) (0.25%).

The Hb solution was gasified with CO gas for 30 minutes, so that the Hb-CO content is more than 98% of the total Hb.

The compositions as indicated in Table 3 were produced by mixing the components of the primary W/O micro-emulsion (base ME) according to Example 1, Table 1, No. 1, No. 2, and 6) and the aforementioned hemoglobin solution, at room temperature, stirring carefully, until a W/O micro-emulsion occurred as a clear gel, or an O/W micro-emulsion occurred as a solution having a particle size of 200 to 300 nm.

TABLE 4
(amount information in ml)

| | 1 | 2 | 3 | 4 | 5 |
|---|---|---|---|---|---|
| Base ME | 6.0 | | | 6.0 | 1.0 |
| No. 1 | | | | | |
| Base ME | | 6.0 | | | |
| No. 2 | | | | | |
| Base ME | | | 6.0 | | |
| No. 6 | | | | | |
| Hemoglobin | 3.0 | | | | |
| solution, | | | | | |
| 30% | | | | | |
| Hemoglobin | | 3.0 | | | 5.0 |
| solution, | | | | | |
| 25% | | | | | |
| Hemoglobin | | | 3.0 | | |
| solution, | | | | | |
| 10% | | | | | |
| Hemoglobin | | | | 3.0 | |
| solution, | | | | | |
| 5% | | | | | |
| Product | Trans- | Transparent | Transparent | Transparent | Solu- |
| form | parent | gel | gel | gel | tion |
| | gel | | | | |

### Example 9

### W/O ME for Skin Treatment With NMF (Water-Binding Substances), Vitamins, and Cosmetics Additives

The compositions were produced by combining primary W/O micro-emulsions, as described, and an NMF solution, stirring carefully, at room temperature, until a clear W/O micro-emulsion having a particle size of 50 to 300 nm is formed. The active substances used were:

Sorbitol (0.5-5.0%), allantoin (0.2-0.5), glycerin (0.5-10.0), PCA-Na (0.5-5.0), urea (0.5-20.0), amino acids or protein hydrolysates (silk protein, wheat germ protein, yeast) (0.1-0.5), oligosaccharides (trehalose, chito-oligosaccharides) (0.1-0.5), water-soluble vitamins and modificates (Vitamin C, Vitamin H) (0.01-0.5), hemoglobin solution, as well as the addition of inorganic salts.

TABLE 4
(information in grams)

| | 1 | 2 | 3 | 4 |
|---|---|---|---|---|
| Base ME No. 2 (Table 1) | 60.0 | | 60.0 | |
| Base ME No. 4 (Table 1) | | 60.0 | | 60.0 |
| Water | 20.8 | 25.75 | 24.6 | 24.8 |
| Sorbitol | 1.0 | 1.0 | 1.0 | 1.0 |
| Allantoin | 0.15 | 0.15 | 0.1 | |
| Pyrrolidone carboxylic acid | 1.0 | 1.0 | 0.5 | 1.0 |
| Na salt | | | | |
| Chito-oligosaccharides | 0.3 | | 1.0 | 1.0 |
| Urea | 1.5 | 1.0 | 1.5 | 1.0 |
| Glycerin | | 0.8 | | |
| Aloe vera, 100% | 0.1 | | 0.3 | 0.3 |
| Ascorbic acid Na | | | 0.01 | |
| Biotin | | | | 0.5 |
| Silk protein | 0.2 | | 0.5 | 0.3 |
| Hydrolysate | | | | |
| Hydrolyzed collagen (crotein D) | | 0.3 | | 0.3 |
| Hyaluronic acid | | 0.01 | | |
| NaCl | 0.1 | | 0.1 | |
| Hemoglobin | | 0.02 | 0.02 | 0.02 |

### Example 10

### O/W Micro-Emulsion for Skin Treatment With NMF (Water-Binding Substances), Vitamins, and Cosmetic Additives

Part of the primary W/O micro-emulsion from Example 9 (No. 1) was mixed with 5 parts water or an aqueous solution. This resulted in a clear, opaque ME solution having a particle size of 100 to 200 nm.

### Example 11

### W/O Micro-Emulsion Containing Plant and (or) Microbiological Extracts

The following aqueous or alcohol extracts or mixtures of them, namely: aloe vera, echinacea, green tea, hammamelis extract, meristern extract, cashew tree, Polyplant ME (Polygon Chemie, Switzerland), were taken as the aqueous phase, separately or as mixtures, and mixed with an oil phase (like No. 1 to No. 5, Table 4).

### Example 12

### Production of an O/W Micro-Emulsion Containing Plant or Microbiological Extracts

Part of the primary W/O micro-emulsion Table 1 (No. 1 from Example 1) was mixed with 5 parts plant extracts and Hb solution. This resulted in a clear, opaque micro-emulsion solution having a particle size of 100 to 200 nm.

### Example 13

### W/O Micro-Emulsion With Polysaccharides and Oligosaccharides

Oligosaccharides (trehalose, chito-oligosaccharides), polysaccharides, hyaluronic acid, chitosan, xanthane, aloe vera) were taken as the aqueous phase and formulated into an ME as in Example 11.

### Example 14

### O/W Micro-Emulsion With Polysaccharides and Oligosaccharides

Part of the primary W/O micro-emulsion from Example 13 was mixed with 5 parts water or aqueous solution and Hb solution. This resulted in a clear, opaque micro-emulsion solution having a particle size of 100 to 200 nm.

### Example 15

### W/O Micro-Emulsion for Self-Tanning

The composition was produced by combining two parts of the primary micro-emulsion (Example 1, Table 1, No. 1) and one part of a 5% aqueous solution of 1,3-dihydroxy propan-2-one.

### Example 16

### W/O Micro-Emulsion for Depigmentation

Here, the following substances were incorporated into a base micro-emulsion according to Example 1, Table 1, No. 1 to 5: hydroquinone and derivatives, acelainic acid, 4-isopropyl catechol, natural substances such as plants rich in ascorbinic acid (parsley, lemon juice, rosehips, iris).

### Example 17

### W/O Micro-Emulsion With Super-Oxide Dismutase and Catalase

Super-oxide dismutase (SOD), recombinant human super-oxide dismutase from yeast (Resbio Ltd. Russia), catalase or hemoglobin/super-oxide dismutase and hemoglobin/catalase were incorporated into a base micro-emulsion according to Example 8, Table 3, No. 1 to 5, instead of hemoglobin solution. The amount of catalase and super-oxide dismutase is from 0.01 to 0.3%.

### Example 18

### W/O Micro-Emulsion With Antioxidants

Melatonin, ascorbic acid, cysteine, and N-acetyl cysteine were worked into a micro-emulsion according to Example Table 3, No. 1 to 5, into the aqueous phase.

### Example 19

### Dermatological/Medical W/O and O/W Micro-Emulsions

Two parts of the primary micro-emulsion according to Example 1, No. 1 to 5, were mixed with one part aqueous solutions containing active substance (Table 5). This resulted in a clear, opaque micro-emulsion solution having a particle size of 100-300 nm.

TABLE 5

| | | Active | |
|---|---|---|---|
| Base ME | Active substance 1 | substance 2 | Use |
| Example 1 No. | Glycerrhitinic | Melatonin | Acne |
| 1 to 5 | acid | | |
| | Zn salt | | |
| Example 1, No. | Glycerrhitinic | Borage oil | Neurodermitis |
| 1 to 5 | acid | | |
| Example 1, No. | Urea | Dead Sea salt | Neurodermitis |
| 1 to 5 | | | |
| Example 1, No. | Dihydroxy acetone | | Vitiligo |
| 1 to 5 | | | |

### Example 20

### Demonstration of the Penetration Depth of Micro-Emulsions Into the Skin

The penetration depth of micro-emulsions according to the invention was determined using a peel method and a model skin. Pigs' ears without skin injuries were picked up from the slaughterhouse immediately after slaughtering. The ears were washed thoroughly with mild soap. Afterwards, they were peeled gently with 2% glucolic acid, over a period of 15 minutes. Subsequently, the skin was neutralized with 2% sodium hydrocarbonate solution and rinsed with water. During this time, the ears were kept at a temperature of 35° C.

The following micro-emulsions were applied to the skin surface of the ears and massaged into the skin for about 1 to 2 minutes:
1. ME containing hemoglobin, in accordance with Example 8, No. 3,
2. W/O ME containing NMF, in accordance with Example 9, No. 1,
3. O/W ME containing NMF, in accordance with Example 10,
4. Control ME in accordance with Example No. 1.

Afterwards, they were allowed to act at 35° C. for about 30 minutes. Subsequently, the residues of the micro-emulsion were washed off with mild soap, and the skin was dried off.

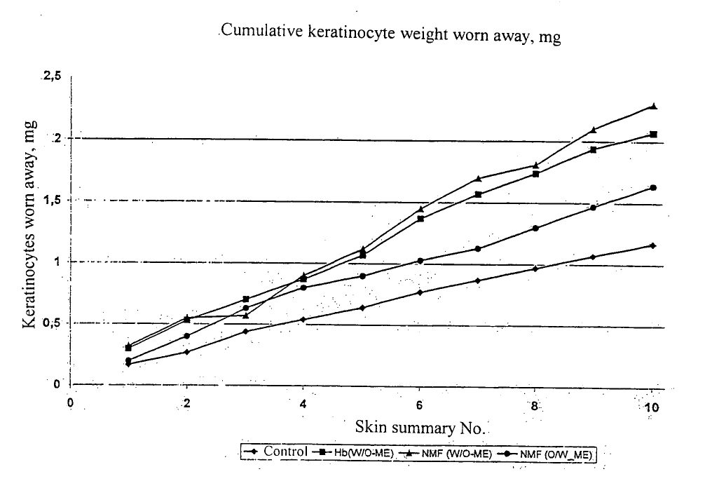

For the peel, 15 Tesafilm strips (Tesafilm, Beiersdorf) were cut to a size of 1.5 cm×1.5 cm and weighed out. The examination fields of 2 cm×2 cm chosen on the pig's ear were covered, one after the other, with the prepared Tesafilm strips, which were then pulled off. The strips were weighed again. The amount of keratinocytes of the stratum corneum that were removed was determined as the difference between the weight of the strips before and after removal. The cumulative weight of keratinocytes worn away was plotted against the removal steps, in a diagram (FIG. 1).

As is evident from this, a significant penetration into the deeper skin layers occurs with the micro-emulsion according to the invention, as such, so that active substances can act better and more effectively.

In the following Example 21, the demonstration of the efficacy of active substances used according to the invention is presented.

### Example 21

### Demonstration of the Facilitated Oxygen Diffusion Into the Skin by Means of W/O Micro-Emulsion Containing Hemoglobin

The demonstration of the facilitated oxygen diffusion into the skin was carried out using the human lower arm. First, the lower arm was cleaned with mild soap. This was followed by a light peel using either 2% glycolic acid or several times with Tesafilm strips (see above).

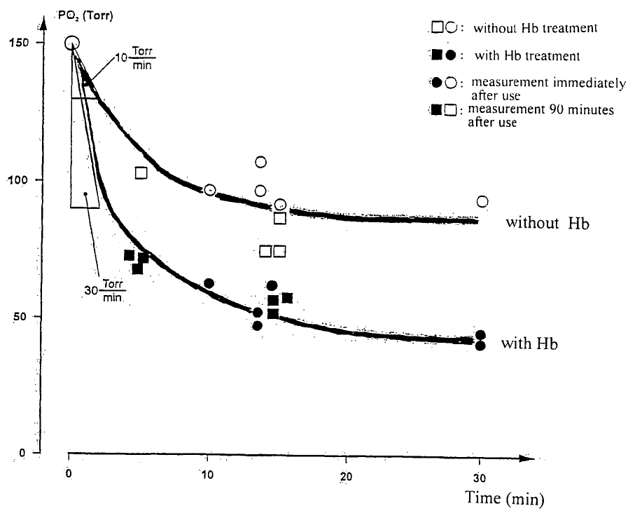

The W/O micro-emulsions containing hemoglobin (No. 3 and No. 5, Example 8) were applied to the prepared skin surface and massaged in lightly, and allowed to act for 15 minutes. As a control, lightly peeled skin was measured. Afterwards, the skin was washed and dried off. At the measurement site, a silicone membrane containing RuCl hexahydrate as the measurement layer was glued on. The decrease in fluorescence at the membrane, as a measure of the oxygen partial pressure pO₂, was continuously measured over the course of 90 minutes, according to the method (M. Stücker, P. Altmeyer et al., The Transepidermal Oxygen Flux from the Environment is in Balance with the Capillary Oxygen Supply, J. Investigative Dermatology 2000, Vol. 114, No. 3, p. 533-540). The oxygen absorption of the living cells in the epidermis was determined as the initial drop of the pO₂ values. FIG. 2 shows the results of these measurements. As is evident from this, the micro-emulsion containing hemoglobin shows a 3 times increase in the oxygen absorption as compared with the control.

### Example 22

### Demonstration of the Skin Tolerance of the W/O Micro-Emulsions Containing Hemoglobin

For the epicutaneous test, which serves to demonstrate a primary skin irritation or a contact allergy, W/O micro-emulsions containing hemoglobin (Example 8, Table 3, No. 3) was used in an amount of 2 to 5 mg/cm² skin. A collective of 32 female and 18 male test subjects aged 18 to 69 years old participated in the test, including 10 test subjects suffering from allergies and 13 test subjects having sensitive skin.

The aforementioned micro-emulsion is applied to the clinically healthy skin and fixed in place using a commercially available test patch. The test patch is removed after 48 hours and the test field is evaluated. A further evaluation takes place after 72 hours.

It was not possible to detect positive or questionable reactions in any of the test subjects, either after 48 hours or after 72 hours, so that there was no indication, in this test, that the micro-emulsion in the present test concentration has a primary irritating effect on the skin. Also, it was not possible to trigger any pre-existing sensitization by means of ingredients of the micro-emulsion in this test.

### Example 23

### Demonstration of the Efficacy of Cosmetic Treatment Using the W/O Micro-Emulsion Containing Hemoglobin

The cosmetic treatment with the W/O micro-emulsion containing hemoglobin (Example 8, Table 3, No. 3) took place over a period of eight weeks, on a total of 14 voluntary female test subjects having dry, slightly aged skin. Before the beginning of the treatment, as well as after the eight weeks of treatment, all of the female test subjects were measured by means of the so-called SELS method (Surface Evaluation of Living Skin). The following skin parameters were measured: roughness, scaliness, smoothness, and wrinkling. The measurements were taken at the forehead, in the region of the corners of the eyes, and in the region of the corners of the mouth.

It was found that in general, a definite skin-smoothing effect was demonstrated in 13 of the female test subjects evaluated. This essentially relates to wrinkling: decrease by 29% (forehead), 43% (corners of the mouth), and 17% (corners of the eye) in comparison with the beginning of treatment. Also, the skin smoothness was improved by 39% (forehead), 72% (corners of the mouth), and 74% (corners of the eye) in comparison with the beginning of treatment.

1. Water-in-oil micro-emulsion, with binary phase differentiability and active substance differentiability, wherein it is free of cross-linking agents and comprises
a) 45 to 90 wt.-% of a liquid oil phase;
b) 5 to 40 wt.-% of a mixture of one or more W/O and one or more O/W surfactants, in a ratio of 1:4 to 1:1.2;
c) 0.01 to 20 wt.-% of one or more emulsifiers;
d) 0.00 to 15 wt.-% of one or more monovalent or bivalent C1-8 alcohols;
e) 1 to 10 wt.-% water or aqueous solutions,
whereby the micelles of this primary micro-emulsion have a particle size of 20 to 400 nm, and the emulsion can be optionally converted to a secondary W/O micro-emulsion or an O/W micro-emulsion, by means of reaction with an aqueous phase.

2. Water-in-oil micro-emulsion as recited in claim 1, wherein it contains 0 to 30 wt.-% of one or more active substances soluble in water or soluble in fat, or mixtures of active substances soluble in water and soluble in fat.

3. Water-in-oil micro-emulsion as recited in claim 1, wherein in addition, 0 to 15 wt.-% additives are present.

4. Water-in-oil micro-emulsion as recited in claim 1, wherein it contains
a) 50 to 80 wt.-% of an oil phase;
b) 5 to 40 wt.-% of a mixture of one or more W/O and one or more O/W surfactants;
c) 0.1 to 10 wt.-% of one or more lecithins, phosphatidyl cholines, or derivatives or mixtures thereof as an emulsifier;
d) 0.0 to 10 wt.-% of one or more monovalent or bivalent C1-8 alcohols;
e) 1 to 10 wt.-% water or aqueous solutions.

5. Water-in-oil micro-emulsion as recited in claim 1, wherein it contains 0.01 to 15 wt.-% alcohol.

6. Water-in-oil micro-emulsion as recited in claim 1, wherein the W/O surfactant(s) is/are non-ionic and have an HLB value of 3 to 7, and that the O/W surfactant(s) is/are non-ionic and have an HLB value of 9 to 18.

7. Water-in-oil micro-emulsion as recited in claim 1, wherein the lecithin, phosphatidyl choline or derivative thereof is selected from among phosphatidyl choline, soy lecithin, egg lecithin, mixtures of phosphatidyl choline and lecithin in various proportions, or mixtures thereof.

8. Water-in-oil micro-emulsion as recited in claim 1, wherein the alcohol is selected from among ethanol, isopropanol, butanol, 1,6-octane diol, 1,2-hexane diol.

9. Water-in-oil micro-emulsion as recited in claim 1, wherein it contains water-soluble active substances, which are selected from among amino acids, peptides, protein hydrolysates, proteins, saccharides, oligosaccharides, polysaccharides and derivatives, hormones and substances similar to hormones, antioxidants, vitamins and pro-vitamins, AHA acids, NMF, oxidants, plant extracts, flavonoids, and plant polyphenols or mixtures thereof.

10. Water-in-oil micro-emulsion as recited in claim 1, wherein it contains fat-soluble active ingredients that are selected from among antioxidants, vitamins and pro-vitamins, unsaturated fatty acids, ceramides, ether oils or mixtures thereof.

11. Water-in-oil micro-emulsion as recited in claim 1, wherein it dermatologically effective agents, selected from among hormones and substances similar to hormones, antimycotics, scar treatment agents, tanning agents, tars, keratinolytics, keratinoplastics, photocumarins, acelainic acid or mixtures thereof.

12. Water-in-oil micro-emulsion as recited in claim 1, wherein electrolytes, oxidants, chelating substances, diffusion reinforcing agents, penetration promoting agents, moisturizers or mixtures thereof are contained as additives.

13. Water-in-oil micro-emulsion as recited in claim 1, wherein it contains biological oxygen carriers, selected from among native, modified, or non-modified hemoglobin, myoglobin, or mixtures thereof, in a total amount of 0.001 to 20 wt.-%.

14. Water-in-oil micro-emulsion as recited in claim 13, wherein it furthermore contains antioxidants, glutathione, super-oxide dismutase, melatonin, flavonoids, amino acids or mixtures thereof.

15. Water-in-oil micro-emulsion as recited in claim 13, wherein it furthermore contains glucose.

16. Water-in-oil micro-emulsion as recited in claim 1, wherein it additionally contains vitamins and pro-vitamins in an amount of 0.01 to 1.0 wt.-%.

17. Water-in-oil micro-emulsion as recited in claim 1, wherein it contains plant extracts in an amount of 0.1 wt.-% to 5 wt.-%, 0.1 wt.-% to 5 wt.-% ether oils, 0.1 wt.-% to 10 wt.-% AHA acids, 0.01 to 0.3 wt.-% hormones or substances similar to hormones, 0.1 to 5 wt.-% essential fatty acids, ceramides, or mixtures thereof.

18. Topically applicable preparation in the form of a micro-emulsion, wherein it represents a composition as recited in claim 1, or a mixture thereof with 0.1 to 90 wt.-% of an aqueous phase, whereby the preparation then contains 1 to 50 wt.-% of the oil phase and a W/O micro-emulsion or an O/W micro-emulsion.

19. Method for the production of a micro-emulsion as recited in claim 1, wherein the oil phase, if applicable with fat-soluble active substances contained in it, containing the surfactant(s), the emulsifier(s), as well as the alcohol(s) and, if applicable, additives, and an aqueous phase, and, if applicable, the water-soluble active substance(s), if applicable additives, are mixed with one another at temperatures from 10 to 30° C., and the primary W/O micro-emulsion obtained in this manner is converted to a secondary W/O micro-emulsion or a secondary O/W micro-emulsion, if necessary with an aqueous phase that can contain additional water-soluble active substances, if applicable, and additives, if applicable.

20. Use of a micro-emulsion as recited in claim 1, as or for the production of a cosmetic, dermatological, pharmaceutical preparation for the topical treatment of skin that has been irritated or damaged or has degenerated due to allergy, bacteria, immunology, external influences, or for the treatment and care of hair.

21. Use as recited in claim 20, wherein skin that has been irritated or changed due to age is treated.

22. Use as recited in claim 21, wherein a preparation is used.

23. Use as recited in claim 20, wherein the emulsion is liquid and is sprayed on or applied as a gel.
